(19) United States
(12) Patent Application Publication
(10) Pub. No.: US 2009/0232784 A1
(43) Pub. Date: Sep. 17, 2009

(54) ENDOTHELIAL PREDECESSOR CELL SEEDED WOUND HEALING SCAFFOLD

(76) Inventors: Dale Feldman, Birmingham, AL (US); Jennifer McCullars, Birningham, AL (US); Jerry Blackwell, Atlanta, GA (US)

Correspondence Address: GIFFORD, KRASS, SPRINKLE,ANDERSON & CITKOWSKI, P.C, PO BOX 7021, TROY, MI, 48007-7021, US

(21) Appl. No.: 11/908,174

(22) PCT Filed: Mar. 10, 2006
(86) PCT No.: PCT/US06/08594

§ 371 Date: Nov. 10, 2008

Related U.S. Application Data
(60) Provisional application No. 60/660,406, filed on Mar. 10, 2005.
(60) Provisional application No. 60/673,589, filed on Apr. 21, 2005.

(51) Int. Cl. A61K 35/12; A61P 17/02; A61K 35/14; A61K 35/407; A61K 35/44

(52) U.S. Cl. 424/93.21; 424/93.7; 424/93.71; 424/93.72; 424/529; 424/553; 424/583

(57) ABSTRACT

Compositions and processes for promoting wound healing are provided by the present invention which increase the rate and completeness of wound healing. Broadly described, an inventive composition includes a degradable scaffold of biocompatible material and a cell involved in wound healing, or a predecessor of such a cell, disposed in contact with the scaffold. In one embodiment, an albumin scaffold is provided with an endothelial cell predecessor disposed therein. Optionally, a cell involved in wound healing or a predecessor of such a cell is administered by local or systemic injection in conjunction with application of an inventive composition. Processes for promoting wound healing are provided as well as processes for producing the described compositions for promoting healing.

### RELATED APPLICATIONS

This application claims priority of U.S. Provisional Patent Application 60/660,406 filed Mar. 10, 2005 and U.S. Provisional Patent Application 60/673, 589 filed Apr. 21, 2005.

### GRANT REFERENCE

Research carried out in connection with this invention was supported in part by CDC grant number R49/CCR403641-15 and NSF grant number 1288803-04-0027. Accordingly, the United States government may have certain rights in the invention.

### FIELD OF THE INVENTION

The present invention relates generally to compositions and methods for promoting wound healing. In particular, the present invention relates to compositions including a degradable scaffold material having a cell involved in the process of wound healing disposed therein.

### BACKGROUND OF THE INVENTION

The process of wound healing is classically described in terms of several phases including an initial inflammatory phase, a subsequent proliferative phase, and a maturation phase. The wound healing process is remarkably consistent and varies little between different tissues. While wound healing is initiated virtually instantaneously following infliction of the wound, the complete wound healing process may take anywhere from days to years in a healthy individual. Further, some types of wounds are known to be resistant to healing, such as bedsores and other types of ulcerations. Delayed healing may also result as a consequence of disease, as in diabetes.

Various cell types are involved in the wound healing process, including endothelial cells, inflammatory cells, platelets, neutrophils, lymphocytes, fibroblasts and epithelial cells. Each of the various cells has an essential role in particular phases of the wound healing process. For instance, in the inflammatory phase of wound healing, the presence of macrophages and lymphocytes is considered to be important in removal of exogenous and/or pathological organisms, as well as extraneous tissue debris, Similarly, certain cell types are of great importance in the proliferative phase of wound healing. In particular, fibroblasts play an important role in producing structural proteins essential for the repair process at a wound site. Endothelial cells are important during this and other phases in promoting revascularization of the wound site. Further, epithelial cells are also important in wound healing, particularly where epithelial integrity has been compromised, such as in a puncture wound or other traumatic wound.

Cells present at a wound site further function to release cytokines and growth factors to attract and activate cells with roles in wound healing. For instance, substances released by macrophages attract fibroblasts and endothelial cells to the wound. However, some individuals may produce fewer cells available to migrate or differentiate at the wound site. In addition, some cells present in an ill patient may be less active in producing chemokines. Such deficiencies can have cascading effects in that the performance failure of a first cell type in a patient results in fewer cells of a second type attracted to a wound site thereby bringing about a significant inhibition of wound healing in the patient.

Various approaches to promoting wound healing have been tried, with limited success. Most of the current wound care products are focusing on artificial skin substitutes. These products are expensive and have to be applied in a surgical setting. Substitute skins do not set up in situ and must be trimmed to fit the wound area. Additionally, most skin substitutes do not increase vascularization of the wound area. Patients with severe wounds typically have two treatment options: surgical treatment followed by six weeks of bedrest, or nonsurgical treatment followed by three to six months of bedrest.

Thus, compositions and methods for promoting wound healing are needed. In particular, compositions and methods for promoting wound healing are needed which provide delivery of one or more types of cell involved in wound healing. The costs associated with these wounds due to treatment, doctor/hospital visits, and lost wages are expected to decrease with more efficient wound healing.

### SUMMARY OF THE INVENTION

A composition for promoting wound healing is provided which includes a degradable scaffold and a wound healing cell type and/or a predecessor cell type to the action of the cell type involved in wound healing. Optionally, the scaffold includes a plasma protein such as albumin and/or a product of an enzymatic modification of a plasma protein such as fibrin. In a particular composition, the plasma protein, or enzymatically modified product thereof, included in the scaffold is isolated from a patient having a wound to be treated.

In one embodiment, the cell involved in wound healing is an endothelial cell, an inflammatory cell, an epithelial cell, a platelet, a neutrophil, a lymphocyte, or a fibroblast. A synergistic effect is noted in formulations where a predecessor cell type to the cell type active in the healing process is included. A predecessor cell type to a wound healing cell type includes an endothelial cell predecessor, an inflammatory cell predecessor, an epithelial cell predecessor, a platelet predecessor, a neutrophil predecessor, a lymphocyte predecessor or a fibroblast predecessor. Combinations of these cells are operative in expediting the healing process. Preferably, the wound healing cell type or predecessor cell type is isolated from a patient having a wound to be treated. Specifically, an endothelial cell predecessor having the surface marker CD34 is provided in the composition. An endothelial cell predecessor having one or more of the surface markers CD133, Flk-1, and Tie-2 is provided to facilitate wound healing.

A composition for promoting wound healing also optionally includes one or more of a bioactive agent such as an antibiotic agent, an anti-inflammatory agent, an analgesic agent, an anesthetic agent, a cytokine, a growth factor or combinations of such agents.

A process for promoting wound healing is provided which includes providing a wound healing cell type or a cell type predecessor to the cell type in addition to providing a degradable scaffold that includes a plasma protein or a product of an enzymatic modification of a plasma protein, disposing the cell type on the scaffold to form a composition for promoting wound healing and applying the composition to a patient's wound. Isolation of the plasma protein, a product of an enzymatic modification of a plasma protein, or the cells distributed in the scaffold are obtained from the patient to receive a wound healing composition is a preferred source of such substances.

A wound healing process optionally includes a composition as described herein that is applied to a wound and a cell type involved in wound healing or a cellular predecessor to the cell type is administered to a patient through a route in addition to the scaffold, for example by local or systemic injection. The cells disposed on the scaffold may be the same or different than the cells administered free of the scaffold.

A process for promoting wound healing includes providing a wound healing cell type or a predecessor cell type that acts prior to the wound healing to a cell type. A cross-linkable component of a degradable scaffold and a cross-linker component of a degradable scaffold are also provided. The cell type is added to the cross-linkable component to yield a first constituent and/or the cell is added to the cross-linker component to yield a second constituent. Depending on the combination, the first constituent and cross-linker component are mixed, the second constituent and the cross-linkable component are mixed, or the first and second constituents are mixed to yield a composition for wound healing. The composition is then applied to a patient's wound. Optionally, a cell type involved in wound healing, a cellular predecessor thereof in healing, or mixture of cells including the cell type involved with wound healing are administered by local or systemic injection. A preferred cell type included in a composition or administered by injection is a CD34+ endothelial cell predecessor.

### BRIEF DESCRIPTION OF THE DRAWINGS

FIG. 1 is an image illustrating full-thickness wounds on rabbit dorsum at the time of surgery;

FIG. 2 is an image of a wound section stained with hematoxylin and eosin in which two vertical lines mark the original wound edges and arrows mark the edges of new epithelial tissue;

FIG. 3 is a graph illustrating epithelialization rate (ER) measurements for injected and non-injected animals having wounds treated with nothing (control), an albumin scaffold, the albumin scaffold loaded with endothelial progenitor cells, and a fibrin scaffold loaded with endothelial progenitor cells;

FIG. 4 is a graph illustrating contraction rate (CR) measurements for two animals having wounds treated as detailed with respect to FIG. 3;

FIG. 5 is a graph illustrating healing rate (HR) measurements for two animals having wounds treated as detailed with respect to FIG. 3;

FIG. 6 is a graph illustrating the ratio of the contraction rate to epithelialization rate (CR/ER ratio) calculated from measurements for two animals having wounds treated as detailed with respect to FIG. 3;

FIG. 7 is a graph illustrating the amount of new tissue that has filled the wound area expressed as “tissue fill percentage” for two animals having wounds treated as detailed with respect to FIG. 3;

FIG. 8 is a graph illustrating the amount of new blood vessel formation in the wound area expressed as “volume fraction” for two animals having wounds treated as detailed with respect to FIG. 3;

FIG. 9A is a digital image showing an untreated wound;

FIG. 9B is a digital image showing a wound treated with a locally applied composition of albumin+rabbit EPCs;

FIG. 9C is a digital image showing a wound treated with a locally applied composition of albumin+rabbit EPCs and with a systemic injection of autologous EPCs;

### DETAILED DESCRIPTION OF THE INVENTION

The present invention has utility in the promotion of wound healing. Compositions and processes are described which increase the rate and completeness of wound healing. Broadly described, an inventive composition includes a degradable scaffold of biocompatible material and a wound healing cell type or a predecessor cell type of the cellular type disposed in contact with the scaffold. Optionally, a wound healing cell type or a predecessor in healing relative to the wound healing cell type is delivered separately from application of an inventive composition.

The term “wound healing cell type” as used herein is intended to mean a cell involved in an inflammatory phase, a proliferative phase, or a maturation phase of wound healing. Such cell types include an endothelial cell; an inflammatory cell; an epithelial cell; a platelet; an immune cell such as a neutrophil or a lymphocyte; and a fibroblast. Also included in an inventive composition is a cellular predecessor in healing relative to the wound healing cell type. The term “predecessor cell type in healing relative to the wound healing cell type” as used herein is intended to mean a cell type which is less than fully differentiated and includes a stem cell, a progenitor cell and/or a precursor cell which can differentiate into a cell involved in wound healing, such as a predecessor to an endothelial cell; an inflammatory cell; an epithelial cell; a platelet; an immune cell such as a neutrophil or a lymphocyte; and a fibroblast. Of particular importance are endothelial cells characterized by the presence of surface markers CD34, CD133, Flk-1 and Tie-2.

The term “degradable” as used herein is intended to mean that a scaffold structure breaks down over time in situ in contact with a wound of a patient such that a composition including the scaffold need not be removed during wound healing or after the wound has healed. Such breakdown is recognized to occur through various processes such as enzymatic or hydrolytic.

Although generally described for use in promoting wound healing in human patients, it is appreciated that inventive processes and compositions may be used to promote wound healing in animals of other species such as dogs, cats, horses, cows, pigs, sheep, goats, fowl, and laboratory animals such as rats, mice, guinea pigs and rabbits.

An inventive cell-seeded scaffold is advantageous in that application of cells contributes to the generation of appropriate tissue structures at the wound site, increasing the rate of the healing process. In addition, application of such cells may increase an available quantity of cytokines and/or growth factors available for attraction and activation of other cells involved in wound healing. Further, application of the cells in association with a scaffold provides a temporary structure for the cells and tissues at the wound site. Preferably, the scaffold degrades as the wound heals so that removal of the scaffold may be unnecessary, thus avoiding disruption of the healing wound.

Inventive compositions and processes are applicable for promoting healing of various forms of wounds including soft tissue wounds involving the skin such as pressure ulcers, venous stasis ulcers, diabetic ulcers, arterial ulcers, trauma wounds, burns, and failing skin grafts. Further, inventive compositions and processes may be used to stimulate wound healing of other soft tissues such as may be involved in internal injuries resulting from trauma or surgery. In addition, inventive compositions and processes may be used to stimulate wound healing of bone such as in osteomyelitis and osteoradionecrosis.

A composition for promoting wound healing is provided which includes a degradable scaffold material and a cell type involved in wound healing. In another embodiment, a composition for promoting wound healing is provided which includes a degradable scaffold material and a predecessor cell type that is a cellular predecessor in healing relative to the cell type.

A preferred degradable scaffold material includes a plasma protein. A particularly preferred plasma protein is an abundant plasma protein such as albumin. Also preferred is an embodiment in which the degradable scaffold material includes a product of an enzymatic modification of a plasma protein. For example, enzymatic modification of fibrinogen is performed to produce fibrin. In such an embodiment, inactive fibrinogen, which is composed of polypeptide pairs designated α, β, and γ linked via disulfide bonds, is subjected to thrombin-mediated hydrolysis. This enzymatic modification of fibrinogen produces fibrin monomers that can aggregate at the wound site. The aggregated fibrin is optionally further cross-linked by factor XIIIa, a transglutaminase.

Plasma protein is typically isolated from more than one human individual and combined to create pooled plasma protein. Preferably, a plasma protein is isolated from a single human individual. Most preferably, the plasma protein is isolated from the patient having a wound to be treated by application of an inventive composition.

A plasma protein may be isolated as a relatively pure preparation in some embodiments. In further embodiments, a plasma protein may be present in less purified form. For instance, albumin present in plasma and/or a plasma fraction may be used.

Similarly, where the scaffold includes a product of an enzymatic modification of a plasma protein, the plasma protein subject to enzymatic modification is readily isolated from a plurality of human individuals or preferably, from a single individual. Preferably, the plasma protein subject to enzymatic modification is isolated from the patient having a wound to be treated by application of an inventive composition. Again, such a plasma protein, such as fibrinogen, may be isolated as a relatively pure preparation in some embodiments. In further embodiments, a plasma protein such as fibrinogen may be present in less purified form. For instance, fibrinogen present in plasma and/or a plasma fraction may be used. In one embodiment, such a plasma fraction includes platelet-rich plasma.

Particular examples of degradable scaffolds include an albumin scaffold, a fibrin scaffold or a combination thereof. A degradable albumin scaffold is formed by mixing equal portions of two solutions to form a cross-linked gel. The first solution contains human serum albumin. The second solution contains the cross-linking agent, illustratively including modified poly(ethylene glycol), PEG, glutaraldehyde or transglutaminase at a concentration of approximately 0.10 g/ml as detailed in U.S. Pat. No. 6,656,496. Still other albumin-based scaffolds are detailed in US 2005/0069589. When the two solutions are mixed in a ratio approaching stoichiometry, a gel forms. The gel can be made porous by adding unmodified PEG particles. This albumin scaffold degrades in vivo by both enzymatic and hydrolytic degradation.

The components which make up the bioadhesive scaffold materials are preferably mixed together and immediately applied and/or used in the desired application. The fibrin is preferably present in a concentration of 1-60 mg/ml final concentration. The albumin is preferably present in an amount ranging from approximately 0.1% to 50% by concentration (0.01-0.50 g/ml). More preferably, the albumin is present in an amount of approximately 1.0%-30% concentration and, most preferably, the albumin is present in an amount ranging from 10-30% concentration. The PEG can be used for three separate structural changes to the protein: to attach factors to the polymer, to cross-link the polymer, or to be used as water soluble beads to create porosity. The PEG to attach factors can be used to get one to six attachment sites per molecule. For fibrinogen, one attachment per molecule is preferred. The PEG cross-linker is present in an amount ranging from approximately 0.1% to 30% by concentration. Preferably, the PEG is present in an amount ranging from between approximately 5% and 25% concentration. The PEG for imparting porosity uses 10-50% by volume of 10-500 μm (micron) PEG beads (5,000-2,000,000 MW). Preferably, the PEG beads are in the 10-20% by volume of 120-200 μm beads (20,000-100,000 MW). It should be noted that the relative amounts of fibrinogen, albumin, cross-linking agent (PEG), and PEG beads may be varied in order to alter the properties of the bioadhesive scaffold material including the curing time and the viscosity. That is, the relative amounts of the components may be altered, for example, in order to tailor the bioadhesive material to a specific application or method of applying the bioadhesive material.

The bioadhesive scaffold materials can be applied by any suitable technique including the use of an applicator, injected via a syringe or sprayed.

A degradable fibrin inventive scaffold is formed by mixing equal portions of two solutions. The first solution contains human fibrinogen (approximately 5-15 mg/ml) and Factor XIII. The second solution contains bovine thrombin (10-100 U/ml) in calcium chloride. When mixed, these solutions also form a degradable gel which is broken down in situ by enzymes. It is appreciated that an interpenetrating network (IPN) is formed by cross-linking in a single volume of albumin and fibrin, simultaneously or sequentially. By combining the first and second solutions of an albumin and fibrin gel system and applying the same to a wound, an IPN is formed. An IPN network tends to afford a longer degradation time than either an albumin or fibrin network alone.

In a particularly preferred embodiment, an endothelial cell predecessor is included in an inventive composition. An endothelial cell predecessor in the form of an endothelial cell precursor, also termed an endothelial progenitor cell, is preferred. Endothelial progenitor cells (EPCs) are a type of stem cell derived from the bone marrow. EPCs are isolated from human bone marrow, peripheral blood, umbilical cord blood, and fetal liver and are characterized by the expression of several surface markers, most importantly CD34, CD133, Flk-1, and Tie-2. EPC cells have the ability to travel to sites of tissue/blood vessel damage in the body. Once there, EPCs differentiate into mature endothelial cells and begin forming new blood vessels, a process known as vasculogenesis. EPCs are isolated from peripheral blood or bone marrow. Techniques for isolation include, for instance, a magnetic cell separation technique. In this technique, magnetic microbeads coated with an antibody to the CD34 marker are used to separate the CD34+ EPCs from the rest of the blood cells as further described in examples below [¹]. ¹Hristov M, Erl W, and Weber P C. Endothelial Progenitor Cells: Mobilization, Differentiation, and Homing. Arterioscler Thromb Vasc Bio. 2003; 23: 1185-1189.

While inventive compositions and methods are generally described as including a purified population of cells, it is appreciated that use of a heterogeneous cell population often has beneficial effects. For example, previous studies have shown CD34− cells play an important role in cell signaling of CD34+ cells. Preferably, a heterogeneous population of cells including EPCs is administered. More preferably, more than one population of isolated cells may be administered, such as CD34+ and CD34− cells. By way of example, total peripheral blood mononuclear cells, a heterogeneous population of cells which includes EPCs, is administered.

Optionally, an inventive composition includes a bioactive agent. The term bioactive agent as used herein is intended to mean an agent having a therapeutic effect on a patient having a wound. Exemplary and preferred therapeutic effects include inhibition of infection, mitigation of pain, inhibition of inflammation and promotion of wound healing. Exemplary bioactive agents include an antibiotic agent, an anti-inflammatory agent, an analgesic agent, an anesthetic agent, a cytokine and a growth factor.

In a further embodiment, the wound healing cell type or predecessor cell type thereto includes an expression construct encoding a bioactive agent. For example, an exogenous or over-expressing endogenous expression construct is included which encodes and expresses at the wound site for example an antibiotic agent, an anti-inflammatory agent, an analgesic agent, an anesthetic agent, a cytokine or a growth factor. Nucleic acid sequences encoding exemplary bioactive agents are described in further detail below.

The construction and use of expression constructs is known to those of skill in the art as is illustrated, for example, in standard molecular biology texts such as Molecular Cloning: A Laboratory Maanual by Sambrook, J. et al., Cold Spring Harbor Laboratory; 3rd edition. Further, details of methods used to introduce an expression construct into a cell are known to those of skill in the art as is also illustrated in texts such as the Sambrook et al. manual referenced above.

A therapeutic treatment may be used in conjunction with compositions and processes for wound healing according to the present invention. Illustratively, such therapeutic treatments include electrostimulation, hydrotherapy, hyperbaric oxygen treatment, negative pressure therapy, and ultrasound treatment. Parameters for such treatments are known in the art, for example as described in Kloth, L. C., Adjunctive interventions for wound healing, Chapter 10 in Kloth, L. C. and McCulloch, J. M., Wound Healing: Alternatives in Management, 3rd Ed., 2002, F. A. Davis Co., Philadelphia, Pa.; and Hess, C. L. et al., Ann. Plast. Surg., 2003, 51:210-218, A review of mechanical adjuncts in wound healing: hydrotherapy, ultrasound, negative pressure therapy, hyperbaric oxygen, and electrostimulation.

Various devices and methods for use of electrostimulation in wound healing therapy are used. Exemplary devices and methods are described in U.S. Pat. Nos. 5,395,398; 6,334,069; 6,907,294; and 6,941,173.

Electrical stimulation of the wound area is performed according to any of various protocols which promote wound healing. For example, stimulation may be performed using a monopolar or bipolar electrode arrangement. Electrical stimulation may include continuous direct current and preferably includes pulsed current stimulation. Pulsed current protocols include relatively low voltage pulsed current protocols, typically using voltages in the range of less than 150 volts, often in the range of about 75-150 volts to deliver a biphasic or monophasic waveform. Higher voltages are often used in relatively high voltage pulsed current stimulation. Such a protocol typically provides a monophasic waveform. Pulse duration varies from about 10 microseconds-500 microseconds. Pulse frequency may be in the range of 50-150 pulses per second. Duration of the stimulation may range from 10 seconds to several hours per day, typically 3-7 days/week. Continuous pulsed stimulation is also possible.

While the above are indicative of exemplary stimulation parameters, it is clear that many different stimulation protocols are useful and effective in wound healing. Further details of parameters involved in clinical electrical stimulation of wound healing may be found in Baker, L. L. et al. Diabetes Care, 20(3):405-412, 1997, Effects of electrical stimulation on wound healing in patients with diabetic ulcers; and Kloth, L. C., Electrical stimulation for wound healing, Chapter 9 in Kloth, L. C. and McCulloch, J. M., Wound Healing: Alternatives in Management, 3rd Ed., 2002, F. A. Davis Co., Philadelphia, Pa.

Advantageously, electrical stimulation may also be used to regulate expression of an expression construct encoding a polypeptide bioactive agent. An expression construct is provided which includes regulatory elements responsive to electrical stimulation so as to increase transcription of a polynucleotide encoding a polypeptide bioactive agent operably linked to such regulatory elements. In embodiments of the present invention, a regulatory element of a gene upregulated by electrical stimulation is operably linked to a polynucleotide encoding a bioactive agent to be expressed.

The term “expression construct” as used herein refers to a recombinant DNA molecule containing a desired nucleic acid coding sequence encoding a bioactive agent and containing appropriate regulatory elements necessary or desirable for the expression of the operably linked coding sequence in a particular cell. The term “regulatory element” as used herein refers to a nucleotide sequence which controls some aspect of the expression of nucleic acid sequences. Exemplary regulatory elements illustratively include an enhancer, an internal ribosome entry site (“IRES”), an origin of replication, a polyadenylation signal, a promoter, a transcription termination sequence, and an upstream regulatory domain, which contribute to the replication, transcription, post-transcriptional processing and/or translation of a coding sequence and/or encoded polypeptide in a cell.

The term “operably linked” as used herein refers to connection of two or more nucleic acid molecules, including a polynucleotide to be transcribed and a regulatory element such as a promoter or an enhancer element, which allows transcription of the polynucleotide to be transcribed.

The term “promoter,” as used herein, refers to a DNA sequence operably linked to a desired coding sequence encoding a bioactive agent which is capable of controlling the transcription of the coding sequence. A promoter is generally positioned upstream of a desired coding sequence encoding a bioactive agent, and provides a site for specific binding by RNA polymerase and other transcription factors.

Regulatory elements may contribute to constitutive or stimulus-regulated transcription or translation. The term “constitutive” when made in reference to a regulatory element is intended to indicate that the regulatory element is capable of directing transcription of an operably linked nucleic acid sequence in the absence of a particular stimulus.

In contrast, a “stimulus-regulated regulatory element” is a regulatory element which controls a level of transcription or translation of an operably linked nucleic acid sequence encoding a polypeptide bioactive agent in the presence of a stimulus. The level of transcription and/or translation in the presence of the stimulus is increased compared to the level of transcription and/or translation of the in the absence of the stimulus. In the context of the present invention, such a stimulus includes electrical stimulation of a cell containing an expression construct encoding a polypeptide bioactive agent. In particular, an expression construct includes one or more regulatory elements of a gene regulated by electrical stimulation such that transcription and/or translation of the encoded polypeptide bioactive agent is increased by electrical stimulation of a cell containing such an expression construct, thereby increasing the level of the bioactive agent. Such a regulatory element is a promoter in a preferred embodiment. In a further preferred embodiment, such a regulatory element is an enhancer

Genes showing upregulated transcription in response to electrical stimulation of cells illustratively include acetylcholinesterase (Sketelj, J., J. Neurosci., 1998, 18(6):1944-52, Acetylcholinesterase mRNA level and synaptic activity in rat muscles depend on nerve-induced pattern of muscle activation; Sketelj, J., J. Neurosci. Res., 1997, 47(1):49-57, Specific impulse patterns regulate acetylcholinesterase activity in skeletal muscles of rats and rabbits; Michel, R. N., J Cell Biol. 1994, 127(4):1061-9, Neural regulation of acetylcholinesterase mRNAs at mammalian neuromuscular synapses.); troponins, such as troponin I; (Rana, Z. A. et al., J. Physiol., 2005, 562(Pt 3):815-28, Imaging transcription in vivo: distinct regulatory effects of fast and slow activity patterns on promoter elements from vertebrate troponin I isoform genes; Calvo, S., Dev. Genet. 1996, 19(2):169-81, Transcriptional control of muscle plasticity: differential regulation of troponin I genes by electrical activity); and potassium channels, such as Kv1.1, Kv1.2, and Kv1.4 (Grosse, G. et al., J. Neurosci., 2000, 20:1869-1882, Expression of Kv1 Potassium Channels in Mouse Hippocampal Primary Cultures: Development and Activity-Dependent Regulation.)

Regulatory elements involved in transcriptional regulation of such genes illustratively include those described in the following references: Li, Y. et al., J. Biol. Chem. 268(5):3563-3572, 1993, Promoter elements and transcriptional control of the mouse acetylcholinesterase gene; Mutero, A. et al., J. Biol. Chem. 1995 270(4):1866-72, Promoter elements of the mouse acetylcholinesterase gene: Transcriptional regulation during muscle differentiation; Ben Aziz-Aloya, R. et al., Proc. Natl. Acad. Sci. USA, 1993, 90(6):2471-5, Expression of a human acetylcholinesterase promoter-reporter construct in developing neuromuscular junctions of Xenopus embryos; Meshorer, E. et al., J. Biol. Chem., 2004, 279(28):29740-51, Combinatorial complexity of 5′ alternative acetylcholinesterase transcripts and protein products; Getman, D. K. et al., J. Biol. Chem., 1995, 270(40):23511-9, Transcription factor repression and activation of the human acetylcholinesterase gene; Shapira, M. et al., Hum. Mol. Genet., 2000, 9(9):1273-81, A transcription-activating polymorphism in the ACHE promoter associated with acute sensitivity to anti-acetylcholinesterases; Corin, S. J. Et al., J. Biol. Chem., 1994, 269(14):10651-9, Structure and expression of the human slow twitch skeletal muscle troponin I gene; Wymore, RS. et al., J. Biol. Chem., 1996, 271(26):15629-34, Characterization of the transcription unit of mouse Kv1.4, a voltage-gated potassium channel gene; Wymore, R. S. et al., Genomics, 1994, 20(2):191-202, Genomic organization, nucleotide sequence, biophysical properties, and localization of the voltage-gated K+channel gene KCNA4/Kv1.4 to mouse chromosome 2/human llpl4 and mapping of KCNC1/Kv3.1 to mouse 7/human 11p14.3-p15.2 and KCNA1/Kv1.1 to human 12p13.

An exemplary expression construct which may be used in compositions and/or methods according to the present invention includes a troponin I slow regulatory element operably linked to an expressable polynucleotide encoding a bioactive agent in an expression construct as described in Dahler, A. et al., Gene. 1994, 145(2):305-10, Expression vectors encoding human growth hormone (hGH) controlled by human muscle-specific promoters: prospects for regulated production of hGH delivered by myoblast transfer or intravenous injection.

An expression vector construct including a regulatory element of a gene characterized by transcription upregulated in response to electrical stimulation also includes a polynucleotide encoding a bioactive agent as mentioned above. Such a bioactive agent may be a cytokine or a growth factor, for example. In a preferred embodiment, such a polynucleotide encodes a bioactive agent such as a cytokine or a growth factor of the same species as a patient in which the construct is to be used in promoting wound healing.

Such cytokines illustratively include interleukins. Examples of interleukins include IL-1, such as polypeptides encoded by nucleotide sequences identified as Genbank Accession Nos. M15330, M28983, E04743 and M15131; IL-2, such as polypeptides encoded by nucleotide sequences identified as Genbank Accession Nos. E01108, K02797; IL-3, such as polypeptides encoded by nucleotide sequences identified as Genbank Accession No. A02046, M14743; IL-4 such as polypeptides encoded by nucleotide sequences identified as Genbank Accession Nos. M13982 and M25892; IL-5, such as polypeptides encoded by nucleotide sequences identified as Genbank Accession Nos. X06270 and J03478; IL-6, such as polypeptides encoded by nucleotide sequences identified as Genbank Accession Nos. E02772 and M20572; IL-7, such as polypeptides encoded by nucleotide sequences identified as Genbank Accession Nos. J04156 and M29054; IL-8, such as polypeptides encoded by nucleotide sequences identified as Genbank Accession No. M28130; IL-9, such as sequences disclosed in Kelleher et al, Blood. 1991; 77: 1436-1441, Immunogenetics 1990; 31(4):265-270); IL-10 such as polypeptides encoded by nucleotide sequences identified as Genbank Accession Nos. M84340 and U16720; IL-11, such as sequences disclosed in Paul et al, Proc Natl Acad Sci USA. 1990; 87: 7512-7516, Morris et al, Exp Hematol. 1996; 24: 1369-1376), IL-12, such as polypeptides encoded by nucleotide sequences identified as Genbank Accession Nos. M86671 and S82412; IL-13, such as polypeptides encoded by nucleotide sequences identified as U31120 and L13028); IL-14, such as sequences disclosed in Ambrus et al, Proceedings of the National Academy of Science (USA), 1993, 90:6330-4); IL-15, such as polypeptides encoded by nucleotide sequences identified as Genbank Accession Nos. AF031167 and U22339); IL-16, such as polypeptides encoded by nucleotide sequences identified as Genbank Accession Nos. AF006001 and M90391), IL-17, such as polypeptides encoded by nucleotide sequences identified as Genbank Accession Nos. U32659 and U43088); IL-18, such as polypeptides encoded by nucleotide sequences identified as Genbank Accession Nos. D49949 and D49950); IL-19, such as polypeptides encoded by nucleotide sequences identified as Genbank Accession No. AY040367); IL-20, such as polypeptides encoded by nucleotide sequences identified as Genbank Accession Nos. NM02130 and NM018724), IL-21, such as polypeptides encoded by nucleotide sequences identified as Genbank Accession Nos. AF254069 and AF254070), IL-22, such as polypeptides encoded by nucleotide sequences identified as Genbank Accession No. AF279437), IL-23, such as polypeptides encoded by nucleotide sequences identified as Genbank Accession Nos. AF301619, AF301620 and AY055379; IL-24, such as polypeptides encoded by nucleotide sequences identified as Genbank Accession Nos. AF276916 and NM053095), IL-25, such as polypeptides encoded by nucleotide sequences identified as Genbank Accession No. NM080837), TNF-alpha such as polypeptides encoded by nucleotide sequences identified as Genbank Accession Nos. M16441 and Y00467), and GM-CSF such as polypeptides encoded by nucleotide sequences identified as Genbank Accession Nos. X03019 and M11220); and species homologs of each of these.

Growth factor bioactive agents include connective tissue growth factor, CTGF, such as encoded by nucleotide sequences identified as Genbank Accession No. NM 001901; epidermal growth factor, EGF, such as encoded by nucleotide sequences identified as Genbank Accession No. BC093731; fibroblast growth factor, FGF, such as encoded by nucleotide sequences identified as Genbank Accession No. M65053; interferons, IFN, such as encoded by nucleotide sequences identified as Genbank Accession No. BC074928; insulin like growth factor, IGF-1, such as encoded by nucleotide sequences identified as Genbank Accession Nos. X56773 and X56774; keratinocyte growth factor (KGF, such as encoded by nucleotide sequences identified as Genbank Accession No. BC010956; platelet derived growth factor, PDGF, such as encoded by nucleotide sequences identified as Genbank Accession No. S62078; tumor necrosis factor, TNF, such as encoded by nucleotide sequences identified as Genbank Accession No. NM 000594; and vascular endothelial cell growth factor, VEGF, such as encoded by nucleotide sequences identified as Genbank Accession No. M95200; and species homologs of each of these.

An expression construct including a regulatory element of a gene whose transcription is upregulated by electrical stimulation may be used to transfect a wound healing cell type and/or a predecessor of a cell type that acts prior to wound healing as described herein. In other embodiments, such an expression vector construct may be transfected into another cell type, preferably an electrically excitable cell, precursor thereto, or derivative thereof, illustratively a muscle cell, including myoblasts and myotubes; muscle-derived cell; such as a muscle-derived cell line including for example a C2C12 cell; and a satellite-cell; a neuron; a neuroblast, and/or a neuron-derived cell or cell line. Transfection of such cells is accomplished by standard methods, such as electroporation, calcium phosphate coprecipitation, lipofection, and injection, as described for instance in Sambrook et al., referenced herein. Such a transfected cell is optionally included in an inventive composition and/or provided for use in a process according to the present invention so as to promote wound healing. Electrostimulation may be performed to regulate expression of the bioactive agent encoded by the expression construct.

### Process for Promoting Wound Healing

One embodiment of an inventive process includes the steps of providing an inventive composition as described herein and applying it to a wound. In a preferred option, a further step includes delivery of a wound healing cell type or a predecessor cell type into the patient having the wound to be treated by a route other than by delivery in conjunction with a scaffold. By way of example, other delivery routes include wound lavage, subcutaneous injection proximal to the wound, intrathecally, and intravenous injection.

The preparation of an inventive composition for wound healing includes mixing a cross-linkable protein solution with a cross-linking agent to form a protein matrix scaffold. As the scaffolds are formed from two solutions, a wound healing cell type alone or in combination with a predecessor cell type is seeded into either the cross-linkable protein solution or the cross-linker solution. Bioactive agents are similarly added to one or the other of the scaffold component solutions. Additionally, porosity enhancing polymeric beads or modifying agents as detailed in U.S. Pat. No. 6,656,496 are similarly added prior to scaffold gelation. The effect of the inventive scaffold is modified by the delivery of a solution containing cells of a wound healing cell type alone or in combination with a predecessor cell type, the delivery being independent of scaffold gelation. Cell type delivered independent of the scaffold need not be the same as that incorporated within the scaffold. Indeed, successive wound healing cell types are appreciated to be operative as deliverants independent of the scaffold in different stages in the wound healing process. As a result, a wound is exposed to a solution of endothelial cells, inflammatory cells, macrophages, or lymphocytes immediately subsequent to the wound in order to enhance inflammatory response and clearance of antigenic substances. Thereafter, as wound healing enters a proliferative phase, fibroblasts and endothelial cells are delivered in the vicinity of the wound. It is appreciated that in order to not disturb the scaffolding then in place, the delivery of wound healing cell types and their predecessors subsequent to application of inventive scaffold composition occurs through injection that is local and/or systemic. Preferably, wound healing cell types are provided by both localized and systemic injection. It is appreciated that regular delivery of wound healing cells or predecessor cell types thereto is facilitated by harvesting a population of such cells and allowing them to multiply ex vivo. Increasing deliverable cell populations ex vivo is particularly well suited in the treatment of an individual who is immunologically challenged, too ill, young or elderly to tolerate cell harvest. As a result of ex vivo cell multiplication, an individual's wound healing capability is enhanced. Additionally, concerns about antigenic response to such cells are reduced by solution delivery of autologous cells back to an individual, as compared to harvesting such cells from a pooled source or from another individual.

As noted above, an adjunctive wound healing therapeutic treatment may be used in conjunction with compositions and processes for wound healing according to the present invention. Illustratively, such therapeutic treatments include electrostimulation, hydrotherapy, hyperbaric oxygen treatment, negative pressure therapy, and ultrasound treatment. Parameters for such treatments are known in the art, for example as described in Kloth, L. C., Adjunctive interventions for wound healing, Chapter 10 in Kloth, L. C. and McCulloch, J. M., Wound Healing: Alternatives in Management, 3rd Ed., 2002, F. A. Davis Co., Philadelphia, Pa.; and Hess, C. L. et al., Ann. Plast. Surg., 2003, 51:210-218, A review of mechanical adjuncts in wound healing: hydrotherapy, ultrasound, negative pressure therapy, hyperbaric oxygen, and electrostimulation.

Embodiments of inventive compositions and methods are illustrated in the following examples. These examples are provided for illustrative purposes and are not intended as limitations on the scope of inventive compositions and methods.

### EXAMPLES

### Example 1

### Separation of Peripheral Blood Mononuclear Cells (PBMC)

Peripheral blood is collected from the donor using 50 ml syringes coated with 1 ml heparin. Between 100-125 ml of blood is usually collected during each draw. 15 ml of phosphate buffered saline (PBS) without calcium or magnesium is added to 50 ml centrifuge tubes. One tube is used for every 20 ml of blood drawn. 20 ml of collected peripheral blood is mixed with PBS in the tubes. 14 ml Histopaque solution (1.077 g/ml, Sigma) is added to the bottom of the tube to separate the blood cells based on a density gradient. The tubes are then spun in a centrifuge at 1800 rpm (450 g), 20° C., and no brake for 24 minutes. The blood separates to form a layer of red blood cells in the bottom of the tube, a layer of clear Histopaque solution, a cloudy band of PBMC, and the remaining yellowish portion of the plasma. The plasma layer is aspirated, then the PBMC band is harvested from the tube using a 10 ml pipette. The harvested PBMC cells are placed into a new 50 ml centrifuge tube. PBS is added to each tube containing the PBMC to give a final volume of 50 ml. The tubes are spun at 1500 rpm (200 g), 20° C. for 20 minutes. The liquid is aspirated from each tube, and 10 ml PBS is then mixed in the tubes with a pipette. The contents of the tubes are combined into a single tube, and PBS is added to give a final volume of 50 ml. A cell count is then obtained and the tube is spun at 1500 rpm (200 g), 20° C. for 8 minutes. The supernatant is aspirated, leaving only a pellet of PBMC.

### Separation of Bone Marrow Cells

Bone marrow is placed in 50 ml tubes containing 5 ml PBS supplemented with 2 mM ethylene diamine tetra acetate (EDTA) or 0.6% ACD-A or 200 U/ml heparin. Cells are released by diluting a quantity of bone marrow in 10× excess of RPMI 1640 (Hyclone) containing 0.02% collagenase B and 100 U/ml DNAse and shaking at room temperature for 45 minutes. Cells are passed through a 30 micron nylon mesh and 35 ml of diluted cell suspension is layered over 15 ml of Ficoll-Paque® (Amersham Biosciences). The preparation is centrifuged for 35 minutes at 400 g at 20° C. in a swingingbucket rotor without braking. The upper layer is aspirated away, leaving the mononuclear cell layer undisturbed at the interphase. The cells at the interphase are collected and washed twice in PBS containing 2 mM EDTA or 0.6% ACD-A. The washed cells are then centrifuged for 10 minutes at 300 g at 20° C. and resuspended in PBS buffer prior to magnetic labeling.

### Example 2

### Separation of CD34+Predecessor Cells

The PBMC pellet of Example 1 is resuspended in PBS for a final volume of 300 microliters per 108 cells. 100 microliters per 108 cells of Human IgG (Miltenyi Biotech, Inc.) is added to the cell suspension. The blocking reagent inhibits unspecific binding, or Fc-receptor mediated binding, of CD34 Microbeads to non-target cells. CD34+ cells are labeled by adding 100 microliters per 108 cells of the CD34 Microbeads (Miltenyi Biotech Inc.) and mixing well. Alternatively, cells are first incubated with a primary anti-CD34 antibody (QBEND/10, Miltenyi Biotech Inc.), which is modified with a hapten, then the CD34+ cells are magnetically labeled with Anti-Hapten MicroBeads (Miltenyi Biotech Inc.). Cells are incubated for 30 minutes in the refrigerator. Incubating for longer may lead to unspecific cell labeling. The cells are then washed with 50 ml MACS® buffer solution (Miltenyi Biotech Inc.) and spun at 1500 rpm for 8 minutes. The MACS® buffer solution is composed of PBS with 0.5% BSA and 2 mM EDTA. The buffer is aspirated, and the cells are resuspended in 1.5 ml MACS® buffer.

The MACS® separation system is set up as according to the manufacturer's directions with the MACS® MultiStand supporting the MACS® separator magnet. The positive selection column (LS) is placed in the magnetic field of the MACS® separator and, a 50 ml centrifuge tube is placed below the column to catch fluid that passes through the column. The column is then washed with 3 ml MACS® buffer. A 30 micron nylon mesh filter is placed on top of the column and wet with MACS® buffer. Cells are passed though the filter to remove clumps. CD34+ cells which are labeled with the magnetic beads are retained in the column while all other cells pass through. The column is then rinsed 3 times with 3 ml MACS® buffer. The column is removed from the MACS® separator and placed on top of a 15 ml centrifuge tube. 5 ml of MACS® buffer is added into the column, and the retained cells are eluted using the plunger that is supplied with the column. The CD34+ cells are spun at 1500 rpm for 8 minutes.

### Example 3

The separation of CD34+ predecessor cells from bone marrow is accomplished by the method described in Example 2.

### Example 4

The separation of Example 2 is repeated with the substitution of the fluorescence cell separation technique FACS for the magnetic cell separation described in Example 2 to yield a similar quantity of CD34+ cells.

### Example 5

### Fluorescent Labeling of CD34+ Cells

In order to view changes in cell morphology under the microscope during cell culture and to track the cells during the animal studies, the isolated cells are labeled with a fluorescent dye. In an example where CellTracker CM-DiI (choloromethylbenzamido dioctadecylindocarbocyanine) is used, the dye-labeled cells are seen as red under the fluorescent microscope. The procedure for DiI labeling includes: diluting DiI powder to a concentration of 50 mg/ml to form a DiI solution. 3 microliters of the DiI solution per ml of cell suspension, in PBS buffer, is added to the isolated cells and the cells are incubated in the dark for 5 minutes at 37° C. and for 15 minutes at 4° C. The cell-DiI solutions are then spun at 1500 rpm for 8 minutes, and the DiI solution is aspirated. The labeled cells are washed with 10 ml PBS and spun again at 1500 rpm for 8 minutes. The PBS is removed, and the cells are resuspended in culture media. The culture media is composed of Media M199, 20% fetal bovine serum, endothelial cell growth supplement from bovine neural tissue (300 micrograms/ml), penicillin (100 U/ml), and streptomycin (100 micrograms/ml). Before using the CD34+ cells, such as in culturing or injecting, the cells are counted to determine how many CD34+ cells have been isolated. Usually about 1% of the PBMC are isolated as CD34+ endothelial predecessor cells.

### Example 6

Endothelial predecessor cells are isolated from human peripheral blood, bone marrow, or cord blood using techniques similar to those in the prior examples. Because CD34 is the major surface marker for these cells, EPCs are conveniently isolated using magnetic beads coated with an antibody to human CD34.

### Example 7

### Isolation of Endothelial Predecessor Cells from Rabbit Blood and Bone Marrow

For in vivo animal studies, peripheral blood is drawn from the rabbits through the ear vein. PBMC are separated from the blood using the same protocol described above. For human blood, CD34+ cells are isolated using an antibody to human CD34. Rabbits also have a similar CD34 marker, but because there is no known antibody to the rabbit marker, isolation of those specific cells is difficult. Thus, isolated total PBMCs are used since this portion of the blood contains the EPCs.

### Example 8

### Preparing Scaffold Materials for Cell Seeding Preparation of Albumin Scaffolds

Albumin containing gels are made by mixing an albumin solution and a PEG solution to form an adhesive PEG-cross-linked albumin scaffold. The albumin solution is prepared by gently dissolving 0.33 g/ml of lyophilized fraction V rabbit albumin in a 0.85% NaCl solution until clear. Albumin solutions are created within 24 hours of use, placed in amber colored bottles to prevent denaturization, and stored in a 4° C. until use. The PEG solution is prepared by solvating 0.1 g/ml of PEG-disuccinimidyl glutarate (PEG-SG2, molecular weight 10 kDa) into basic N-2-Hydroxyethylpiperazine-N′-2-ethanesulfonic acid (HEPES) solution with a pH of 9.2 until clear. The HEPES buffer is mixed by combining 11.93 g HEPES, 7.03 g NaCl, 0.38 g KCl, and 0.432 g MgCl2.6H2O with distilled water in a 1,000 ml volumetric flask and altering the pH with HCl and NaOH. The albumin gels are formed by combining equal amounts of albumin solution with PEG solution using a dual syringe system.

Preparation of Fibrin Scaffolds

Fibrin gels are made by mixing equal amounts of a fibrinogen solution and a thrombin/calcium chloride solution using a dual syringe system. The fibrinogen solution is prepared by mixing 0.555 ml of rabbit fibrinogen solution with 4.5 ml of TBS for a final concentration of 5 mg/ml fibrinogen. The thrombin solution is prepared by mixing 17 microliters of rabbit thrombin with 200 microliters of 2M CaCl2 and 4.8 ml TBS for a final concentration of 5 U/ml thrombin.

Preparation of Albumin-Fibrin Scaffold

The albumin and fibrinogen solutions as described above in Example 8 are mixed to form a protein precursor solution. Likewise, the PEG and thrombin/calcium chloride solutions as described above are mixed to form a cross-linker solution. The protein precursor solution and cross-linker solutions are loaded into a dual barrel syringe and delivered in equal parts.

### Example 9

### Seeding EPCs into Scaffolds

For seeding into a scaffold, isolated EPCs are mixed into one of the two scaffold solutions. For albumin gels, the cells are preferably added to the albumin protein solution. For fibrin gels, the cells are preferably added to the fibrinogen solution. The gels are formed by mixing the proteinaceous and cross-linker solutions together in equal amounts per Example 8. For cell culture experiments, the scaffolds are allowed to cure, and media is added on top to provide nourishment. This method consistently allowed the cells to survive and proliferate within the gel, and also created scaffolds with cells seeded evenly throughout.

### Example 10

### Methodology for Animal Study I

Endothelial predecessor cell-seeded scaffolds are evaluated in the rabbit dorsal model.

For the study, two animals are used to evaluate the fibrin and albumin scaffolds. Each animal has four wounds; three of the wounds receive treatment with a scaffold, and one wound receives no treatment. The animals are sacrificed after three weeks.

The day prior to surgery, 20-30 milliliters of blood is drawn from the animals through the ear vein. The animals are first given an injection of acepromazine (12 mg/kg), a vasodilator. Then the dorsi of the animals are shaved, and a depilatory cream is applied to remove any remaining hair. Endothelial predecessor cells are isolated from the blood or bone marrow following the techniques outlined in the previous sections.

Animals are anesthetized with ketamine (44 mg/kg) and rompun (5-10 g/kg) administered intramuscularly. Four full-thickness wounds (4×4 cm) are made on the dorsum of each rabbit down to the panniculous carnosus (cutaneous trunci muscle). The four wounds are treated as follows:

A. no treatment (control)

B. treatment with albumin scaffold (no cells)

C. treatment with albumin scaffold+rabbit EPCs

D. treatment with fibrin scaffold+rabbit EPCs

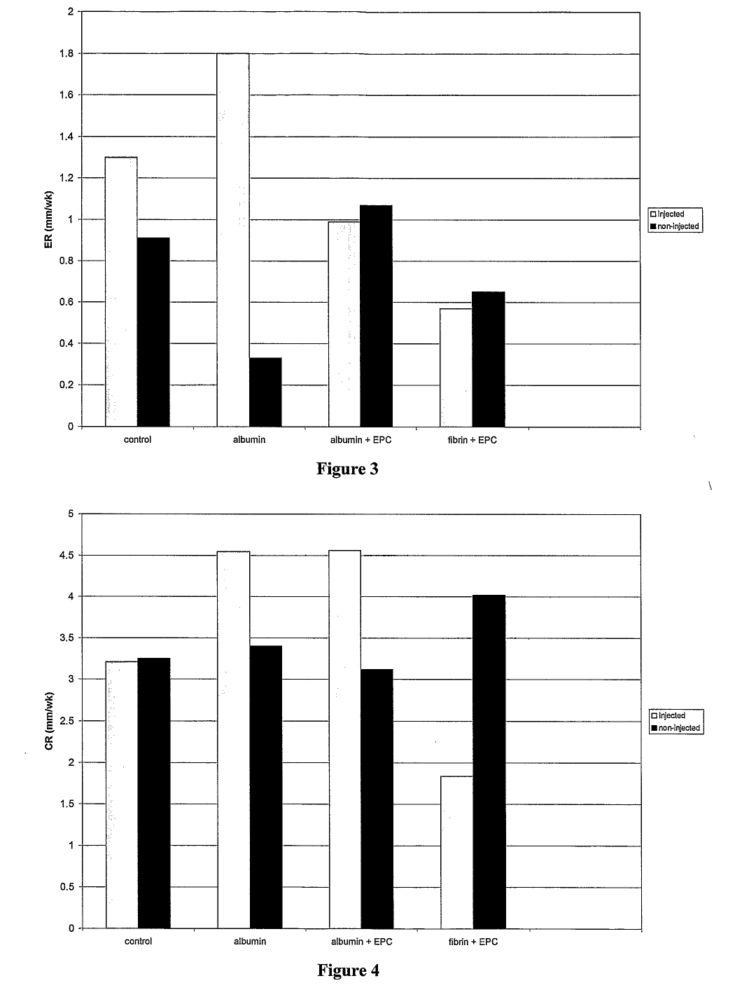

Data from these experiments are shown in graphical FIGS. 3-8 as described below.

Additionally, one of the animals receives an injection of autologous rabbit EPCs (0.5 ml) administered intravenously through the ear vein. The injection is given immediately following surgery. Digital pictures are taken before and after application of the scaffolds, and each wound is covered with an occlusive dressing (Tegaderm).

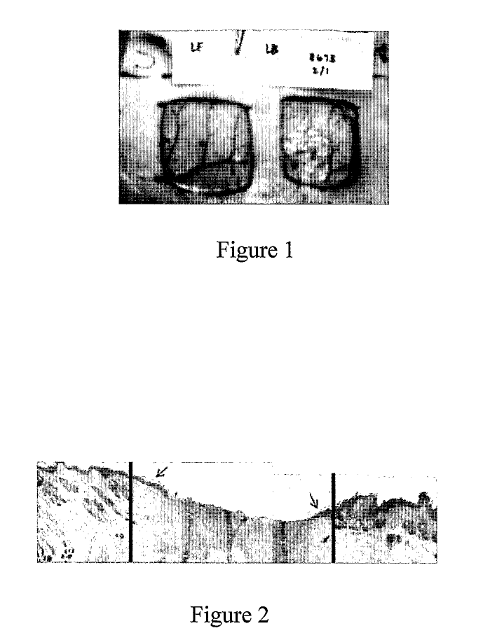

A digital photo showing full-thickness wounds on rabbit dorsum at the time of surgery is shown in FIG. 1.

Following surgery, the animals are observed until conscious. All animals are monitored until they are fully awake and alert. Then they are returned to their cages and checked every day. Analgesics are administered for two days postoperatively (Tylenol 200 mg/kg orally).

Digital pictures are taken and the dressings changed once a week until sacrifice. On the day of sacrifice, the animals are sedated and a digital picture taken to determine contraction rate and epithelialization rate. The animals are sacrificed with an overdose of pentobarbital (100 mg/kg) administered intravenously. After sacrifice, all wounds are excised and prepared for histology evaluation.

### Example 11

### Histomorphometry

Wound samples are fixed in neutral buffered formalin solution, embedded in paraffin, and sectioned at 5 microns in thickness. Sections are stained with hematoxylin and eosin and modified trichrome.

The volume fractions of blood vessels are measured using image analysis software.

Using histology slides, epithelialization rate, contraction rate, and tissue fill percentage are calculated. The wound length is determined by measuring the distance between hair follicles. Contraction rate represents the change in wound size, using the wound margin, due to centripetal contraction. It is calculated as (original wound length−new wound length)/2 time. Epithelialization rate is the growth of new epithelial tissue from the wound edge. It is calculated as length of new epithelium/2 time. Healing rate is calculated by adding the epithelialization rate and contraction rate.

FIG. 2 shows a wound section stained with hematoxylin and eosin. Lines mark the original wound edges, distinguished by hair follicles. Epithelialization rate is calculated by measuring the length of new epithelial tissue (the top layer of tissue) between the lines (arrows). Contraction rate is calculated by comparing the original wound length to the final wound length (the distance between the lines). Tissue fill percentage is calculated by measuring the amount of new tissue filling the wound (between the lines and above the muscle layer).

### Example 12

### Healing Rate

Using the digital images of the wounds and image analysis software, the healing rate (HR) of the wounds for each week is calculated by: HR=|ΔSA|/(P)·(Δt) where SA is surface area, P is perimeter, and t is time.

### Example 13

### Results from Animal Study I

Epithelialization Rate

FIG. 3 shows the epithelialization rate measurements for the two animals in a first study, one animal with injection of autologous EPCs in addition to local treatment with a scaffold+/−EPC composition and one animal treated with a scaffold+/−EPC composition but without injection of autologous EPCs. Epithelialization rate (ER) is shown in mm/wk. In the non-injected animal, the albumin scaffold+EPCs increase ER relative to the non-injection control. In the injected animal, the albumin scaffold alone, the albumin scaffold+EPCs, and the control (injection alone) all improve ER relative to the non-injection control.

Contraction Rate

FIG. 4 shows the contraction rates for all wounds in two animals. Contraction rate (CR) is shown in mm/wk. Fibrin+EPCs treatment in the animal injected with autologous EPCs dramatically decreased CR relative to the non-injection control. Both the injected control and the albumin+EPCs without injection of autologous EPCs decreased CR relative to the non-injection control.

Healing Rate

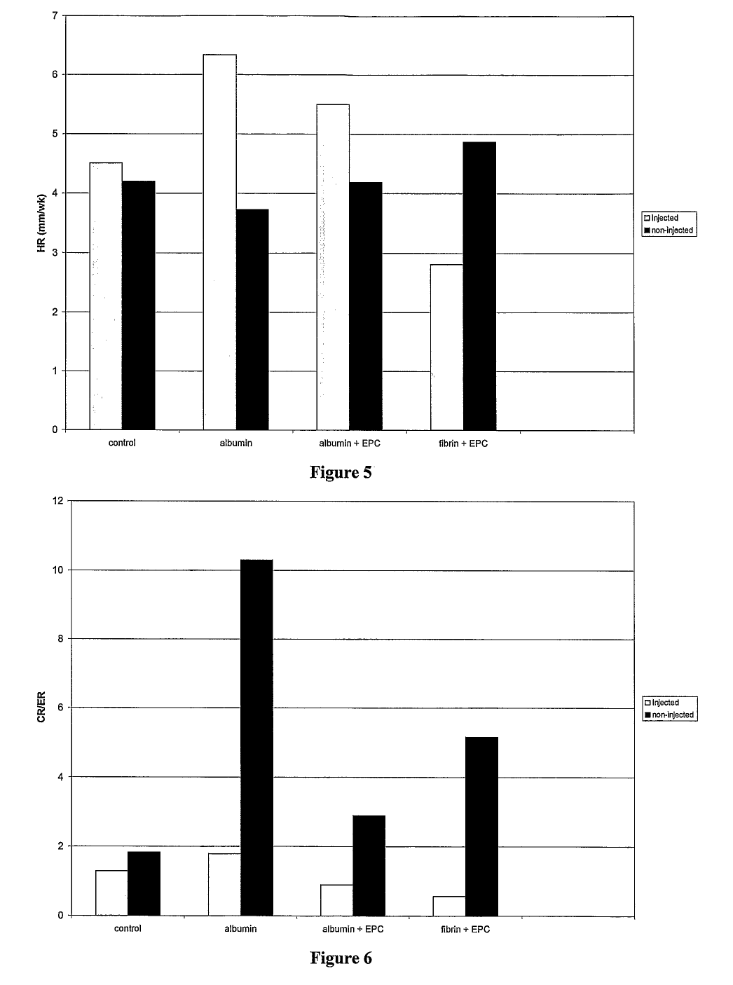

FIG. 5 shows the healing rates (HR) in mm/wk. Healing rates are calculated by combining the ER and CR. The healing rate results show that the fibrin+EPCs increased HR over the control (HR=4.20 mm/wk) in the animal non-injected with autologous EPCs. In the injected animal, the control (injection only), and both albumin treatments (with and without cells) increased HR relative to the non-injection control.

Ratio of CR to ER

In order to relate the epithelialization rate and the contraction rate, the ratio between CR and ER is calculated. Compared to the non-injection control, successful treatment decreases the CR/ER ratio. FIG. 6 shows these ratios for a study of two animals. In the animal not injected with autologous EPCs, the albumin scaffold+EPCs decreased the ratio compared to the control (ratio=3.64). In the animal injected with autologous EPCs, all four treatments reduced the ratio dramatically.

Tissue Fill Percentage

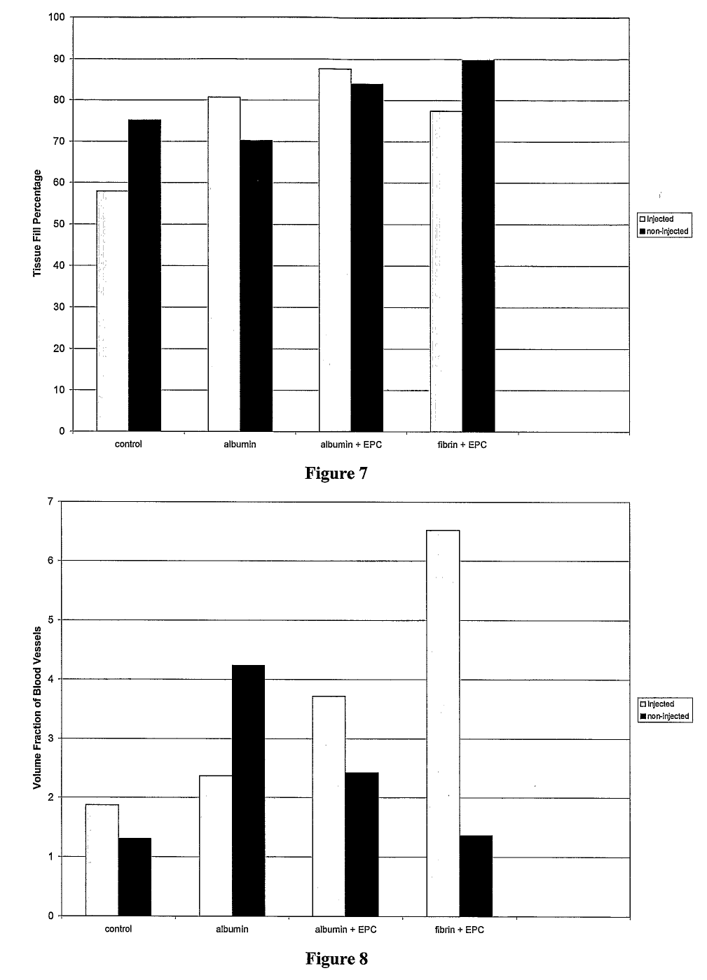

Tissue fill percentage is a measure of the amount of new tissue that has filled the wound area. Increased amount of tissue fill as compared to the non-injection control (TF %=75.21) is indicative of beneficial effect of treatment. From FIG. 7, which shows tissue fill results, it can be seen that both albumin treatments and the fibrin treatment increased tissue fill in the animal injected with autologous EPCs. In the non-injected animal, the albumin+EPCs and fibrin+EPCs increased tissue fill.

Blood Vessels

Another important measure of healing is the vascularization of the wound area, as perfusion is associated with improved healing. FIG. 8 shows the volume percent of blood vessels increased for all treatments except fibrin+EPCs without injection with autologous EPCs.

### Example 14

### Methodology for Animal Study II

This study evaluated the use of albumin scaffolds and EPCs in twenty animals. The methodology is the same as in Example 10 (the first animal study) except that the wounds are treated with different scaffolds. Each animal receives four wounds (4 cm×4 cm):

A. no treatment (control)

B. treatment with albumin scaffold (no cells)

C. treatment with albumin scaffold+rabbit EPCs

D. treatment with albumin scaffold+human EPCs

Additionally, ten of the animals receive an injection of autologous rabbit EPCs. The animals are sacrificed after two and three weeks, with ten animals in each group. (Five injected, five non-injected per time period).

Because specific EPCs cannot be isolated in the rabbit, a population of PBMCs including EPCs are used. In addition, isolated human CD34+ cells are used. Thus, a population of heterogeneous cells including CD34+ cells is compared to a population of isolated CD34+ cells to determine if the purity of the EPC population affects wound healing. Human EPCs are isolated from donor blood using the procedure outlined in Example 2. The rabbit and human cells are labeled with different colored fluorescent dyes (red and green, respectively) to distinguish between them during histological analysis.

At two and three weeks, the animals are sacrificed, the wounds excised and prepared for histological examination. Analysis is performed as in Examples 11 and 12.

### Example 15

### Results from Animal Study II—Non-Injected

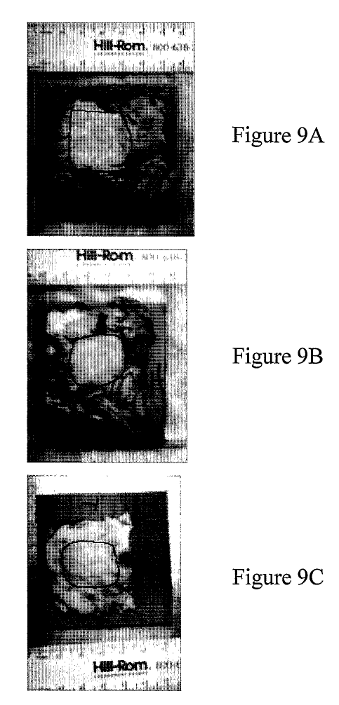

FIGS. 9A, 9B and 9C show digital pictures of wounds with the three different pictures taken at the time of sacrifices (two weeks). The wound edges are outlined. FIG. 9A shows a wound with no treatment (control). Using the equation for calculating overall healing rate given in Example 12, this control wound had a healing rate of 1.04 mm/wk. FIG. 9B shows a wound treated with albumin+rabbit EPCs. For this wound the overall healing rate was 3.79 mm/wk. FIG. 9C shows a wound treated with albumin+rabbit EPCs+injection. This wound had an overall healing rate of 3.19 mm/wk.

Tables I and II

Table I. has the healing rate parameters at 2 weeks for the non injected animals

TABLE I
Healing Rate Parameter Measurements at 2 Weeks

| | | | Albumin + | Albumin + |
|---|---|---|---|---|
| | Control | Albumin | Rabbit PBMC | Human EPC |
| ER (mm/wk) | 1.18 ± 0.34 | 0.99 ± 0.26 | 1.36 ± 0.23 | 1.18 ± 0.36 |
| CR (mm/wk) | 2.03 ± 1.26 | 3.18 ± 1.98 | 3.33 ± 0.82 | 2.11 ± 2.14 |
| HR (mm/wk) | 3.20 ± 0.99 | 4.17 ± 2.17 | 4.17 ± 0.78 | 3.29 ± 2.24 |
| CR/ER Ratio | 2.08 ± 1.75 | 3.00 ± 1.79 | 2.52 ± 0.85 | 1.77 ± 2.18 |
| TF (%) | 86.39 ± 2.66 | 82.39 ± 4.84 | 85.89 ± 5.17 | 84.02 ± 1.10 |

Data shown are mean ± standard deviation.

Table II. has the healing rate parameters at 3 weeks for the non injected animals

TABLE II
Healing Rate Parameter Measurements at 3 Weeks

| | | | Albumin + | Albumin + |
|---|---|---|---|---|
| | Control | Albumin | Rabbit PBMC | Human EPC |
| ER (mm/wk) | 1.39 ± 0.31 | 1.55 ± 0.34 | 1.69 ± 0.68 | 1.10 ± 0.63 |
| CR (mm/wk) | 3.68 ± 1.42 | 3.75 ± 1.21 | 3.89 ± 0.68 | 3.80 ± 0.49 |
| HR (mm/wk) | 5.08 ± 1.65 | 5.30 ± 1.51 | 5.58 ± 1.05 | 4.91 ± 0.91 |
| CR/ER Ratio | 2.61 ± 0.67 | 2.40 ± 0.59 | 2.58 ± 0.98 | 4.20 ± 1.90 |
| TF (%) | 89.39 ± 7.15 | 86.59 ± 9.17 | 94.05 ± 4.38 | 89.40 ± 8.88 |

Data shown are mean ± standard deviation.

Epithelialization Rate (ER)—Tables I and II

At 2 weeks, there was a significant increase in epithelialization rate (ER) for AR (albumin+autologous rabbit PBMC) compared to A (albumin) (37.4% increase, p=0.020). At 3 weeks, both A and AR increased over C (control), but only A increased significantly (11.5% increase, p=0.043), although AR had the higher percent increase (21.6%). A also gave a significantly higher ER than AH (albumin+human CD34+EPCs) at 3 weeks (40.9% increase, p=0.041) and was the only treatment to increase ER significantly between 2 and 3 weeks (56.6% increase, p=0.009). Also at 3 weeks, AH had a lower ER than C, but was not statistically significant (20.9% decrease, p=0.076). Epithelialization rates generally increased between the 2 and 3-week periods (23.0% average increase, p=0.069).

Contraction Rate (CR)—Tables I and II

There were no statistically significant differences in contraction rate (CR) between any of the treatments at 2 or 3 weeks (Tables I and II). At 2 weeks, however, AR had a 64% increase in CR compared to C (p=0.064). There were significant increases in CR between 2 and 3 weeks (49.0% average increase, p=0.011), with C increasing the most (81.3% increase, p=0.043).

Healing Rate (HR)—Tables I and II

At 2 weeks, AR increased healing rate (HR) significantly over C (30.3% increase, p=0.015). There were no significant differences between treatments at 3 weeks. However, all treatments increased HR between 2 and 3 weeks (42.2% average increase, p=0.005), with the biggest increase for C (58.8% increase, p=0.030).

Ratio of CR to ER (CR/ER)—Tables I and II

At 2 weeks, there were no statistically significant differences between treatments. Only AH decreased the ratio compared to C. However, the ratio for AH increased significantly between 2 and 3 weeks (137.2% increase, p=0.049) and was 61% higher than C at 3 weeks (p=0.086).

Tissue Fill Percentage (TF %)—Tables I and II

Tissue fill percentage measures the amount of new tissue that has filled up the wound, with an increase in tissue fill indicative of healing. At 2 weeks, there were no significant differences in tissue fill (TF). However, A and AH both decreased TF compared to C (4.6% decrease, p=0.104 and 2.7% decrease, p=0.058, respectively). At 3 weeks, AR increased TF over A (8.6% increase, p=0.021) and was the best overall treatment. The TF significantly increased between the 2- and 3-week time periods (6.1% average increase, p=0.009) with AR having the largest increase at 9.5% (p=0.014). AH increased 6.4%, but this was not statistically significantly (p=0.108).

Tables III and IV

Table III. has the volume fraction of cells and blood vessels for the non-injected animals at 2 weeks.

TABLE III
Volume Fraction Measurements at 2 Weeks

| | | | Albumin + | Albumin + |
|---|---|---|---|---|
| | Control | Albumin | Rabbit PBMC | Human EPC |
| BVv - inner | 4.30 ± 0.66 | 3.27 ± 0.89 | 3.33 ± 0.91 | 3.09 ± 1.14 |
| BVv - outer | 3.70 ± 0.84 | 4.30 ± 1.29 | 4.48 ± 0.78 | 3.82 ± 1.15 |
| Fv - inner | 23.33 ± 8.86 | 28.48 ± 2.64 | 27.94 ± 5.04 | 20.06 ± 7.49 |
| Fv - outer | 15.58 ± 4.34 | 20.18 ± 3.45 | 13.21 ± 5.54 | 14.91 ± 4.75 |
| Mv - inner | 1.29 ± 1.46 | 1.33 ± 0.51 | 0.85 ± 0.25 | 1.03 ± 0.35 |
| Mv - outer | 1.76 ± 0.54 | 1.51 ± 0.30 | 1.33 ± 0.59 | 1.03 ± 0.41 |
| Nv - inner | 3.45 ± 1.26 | 3.76 ± 0.79 | 3.52 ± 0.85 | 4.42 ± 1.20 |
| Nv - outer | 5.76 ± 1.05 | 4.73 ± 1.33 | 4.97 ± 2.20 | 4.06 ± 1.82 |

Data shown are mean ± standard deviation.

Table IV. has the volume fraction of cells and blood vessels for the non-injected animals at 3 weeks.

TABLE IV
Volume Fraction Measurements at 3 Weeks

| | | | Albumin + | Albumin + |
|---|---|---|---|---|
| | Control | Albumin | Rabbit PBMC | Human EPC |
| BVv - inner | 3.70 ± 1.71 | 4.12 ± 0.79 | 4.18 ± 2.00 | 4.24 ± 1.29 |
| BVv - outer | 4.12 ± 0.95 | 4.79 ± 0.92 | 3.52 ± 1.15 | 4.24 ± 1.50 |
| Fv - inner | 52.00 ± 4.60 | 50.06 ± 7.38 | 49.45 ± 3.73 | 44.85 ± 8.10 |
| Fv - outer | 48.18 ± 6.27 | 51.45 ± 7.44 | 51.27 ± 6.50 | 47.64 ± 5.32 |
| Mv - inner | 0.67 ± 0.25 | 0.85 ± 0.75 | 0.73 ± 0.17 | 0.61 ± 0.30 |
| Mv - outer | 0.73 ± 0.35 | 0.48 ± 0.17 | 0.61 ± 0.30 | 0.79 ± 0.35 |
| Nv - inner | 1.21 ± 0.37 | 1.33 ± 1.02 | 1.64 ± 0.46 | 1.27 ± 0.14 |
| Nv - outer | 1.39 ± 0.46 | 0.85 ± 0.14 | 1.33 ± 0.46 | 1.64 ± 0.66 |

Data shown are mean ± standard deviation.

Blood Vessels (BV)—Tables III and IV

In the inner region, all treatments had a lower volume fraction of blood vessels (BVᵥ) compared to C at 2 weeks, but not significantly—with AR having the largest difference (22.6% decrease, p=0.099). There were no significant differences in the inner region at 3 weeks. However, all treatments except C lead to increases in BVᵥ between the two time periods, though none of the increases were statistically significant.

In the outer region, there were no significant differences in BVᵥ at either time period. However, at 2 weeks, both A and AR increased BVᵥ compared to C. At 3 weeks A was the only treatment to increase BVᵥ compared to C (16.3% increase, p=0.086). A had a significantly higher BVᵥ in the outer region than the inner region at 3 weeks (16.3% increase, p=0.004), and AR was higher in the outer region at 2 weeks (34.5% increase, p=0.060), though not statistically significant.

### Fibroblasts (F)—Tables III and IV

At 2 weeks in the inner region, the volume fraction of fibroblasts (Fᵥ) was significantly lower for AH when compared to A (29.6% decrease, p=0.028). In the outer region, Fᵥ was significantly higher for A than AR (52.8% increase, p=0.003). A was also greater than C and AH, though not significantly (22.8% increase, p=0.077 and 26.1% increase, p=0.065, respectively). There was a significantly lower (Fᵥ) in the outer region compared to the inner region at 2 weeks for the following treatments: C (33.2% decrease, p=0.038), A (29.1% decrease, p=0.006), and AR (52.7% decrease, p=0.005). At 3 weeks, there were no significant differences between treatments in either region. There were significantly higher Fᵥ at 3 weeks than 2 weeks, for both the inner (99.0% average increase, p<0.001) and outer regions (218.0% average increase, p<0.001).

### Macrophages (M)—Tables III and IV

At 2 weeks, in both regions, AH and AR produced the lowest volume fraction of macrophages (Mᵥ). In the outer region, AH was significantly lower than C (41.5% decrease, p=0.016) and also lower than A (31.8% decrease, p=0.035). At 3 weeks, there were no significant differences between any of the treatments in either region. However, there were significant decreases in Mᵥ between 2 and 3 weeks for both the inner (34.8% average decrease, p=0.002) and outer regions (51.0% average decrease, p<0.001).

### Neutrophils (N)—Tables III and IV

At 2 weeks, in the inner region, there was a significant increase in volume fraction of neutrophils (Nᵥ) for AH compared to C (28.1% increase, p=0.035). However, AH was significantly lower than C in the outer region (29.5% decrease, p=0.036). Also, in the outer region, A was lower than C (17.9% decrease), though not significantly (p=0.072). C was significantly higher in the outer region (67.0% increase, p=0.020) than in the inner region at week 2. At 3 weeks, there were no significant differences in the inner or outer region, but A was lower than both C and AR in the outer region (38.8% decrease, p=0.088 and 48.2% decrease, p=0.099, respectively). However, all of the treatments significantly decreased N, between 2 and 3 weeks (67.3% average decrease, p<0.001 for inner region; 72.2% average decrease, p<0.001).

### Example 16

### Results from Animal Study II—Injected with Cells

Tables V and VI

Table V has the healing rate parameters at 2 weeks for the injected animals.

TABLE V
Healing rate parameter measurements at 2 weeks

| | | | | Albumin + | Albumin + |
|---|---|---|---|---|---|
| | | | | Rabbit | Human |
| | | | Albumin + | PBMC + | EPC + |
| | Control | Injection | Injection | Injection | Injection |
| ER (mm/wk) | 1.18 ± 0.34 | 1.75 ± 0.92 | 1.51 ± 0.67 | 1.29 ± 0.13 | 1.45 ± 0.41 |
| CR (mm/wk) | 2.03 ± 1.26 | 4.39 ± 3.07 | 2.31 ± 2.00 | 1.22 ± 2.41 | 2.68 ± 1.79 |
| HR (mm/wk) | 3.20 ± 0.99 | 6.14 ± 3.24 | 3.83 ± 2.17 | 2.51 ± 2.36 | 4.13 ± 1.90 |
| CR/ER Ratio | 2.08 ± 1.75 | 2.55 ± 2.05 | 1.84 ± 2.07 | 1.02 ± 2.07 | 1.90 ± 1.16 |
| TF (%) | 86.39 ± 2.66 | 80.94 ± 4.78 | 83.18 ± 6.79 | 87.87 ± 4.58 | 84.64 ± 5.52 |

Data shown are mean ± standard deviation.

Table VI has the healing rate parameters at 3 weeks for the injected animals.

TABLE VI
Healing rate parameter measurements at 3 weeks.

| | | | | Albumin + | Albumin + |
|---|---|---|---|---|---|
| | | | | Rabbit | Human |
| | | | Albumin + | PBMC + | EPC + |
| | Control | Injection | Injection | Injection | Injection |
| ER (mm/wk) | 1.39 ± 0.31 | 1.38 ± 0.59 | 1.31 ± 0.56 | 1.37 ± 0.59 | 1.37 ± 0.54 |
| CR (mm/wk) | 3.68 ± 1.42 | 4.31 ± 1.50 | 2.66 ± 1.43 | 3.16 ± 1.08 | 3.49 ± 0.86 |
| HR (mm/wk) | 5.08 ± 1.65 | 5.69 ± 1.58 | 4.06 ± 1.85 | 4.53 ± 1.50 | 4.86 ± 0.76 |
| CR/ER Ratio | 2.61 ± 0.67 | 3.56 ± 1.82 | 1.91 ± 0.67 | 2.53 ± 0.76 | 2.97 ± 1.44 |
| TF (%) | 89.39 ± 7.15 | 91.91 ± 6.91 | 86.46 ± 2.50 | 89.80 ± 3.69 | 90.32 ± 3.06 |

Data shown are mean ± standard deviation.

### Epithelialization, Contraction, and Healing Rates—Tables V and VI

For ER, there were no significant differences between the five treatments at either 2 or 3 weeks (Tables V and VI). Additionally, there were no differences between the two time periods. However, AI (albumin+injection) produced a significantly lower CR than 1 (injection alone) at both 2 weeks (47.4% decrease, P=0.044) and 3 weeks (38.3% decrease, P=0.042). Again, there were no differences in CR between the 2- and 3-week periods. Looking at the ratio of CR to ER, there were no differences between treatments at 2 weeks, but again, AI lead to a significantly lower ratio than 1 at 3 weeks (46.3% decrease, P=0.042). Overall, there was a significant increase in the CR/ER ratio between the time periods (54.6% average increase, P=0.026). At 2 weeks (Table V), AI gave significantly lower HR than the injection alone (I, 37.6% decrease, P=0.045), but there were no significant differences between the treatments at 3 weeks (Table VI). The control (C) lead to higher HR at 3 weeks than at 2 weeks (58.8% increase, P=0.030).

### Tissue Fill Percentage (TF %)—Tables V and VI

At 2 weeks (Table V), I produced significantly lower TF than C (6.3% decrease, P=0.028) and ARI (7.9% decrease, P=0.050). There were no differences between treatments at 3 weeks (Table VI). Overall, TF increased between 2 and 3 weeks (6.0% average increase, P=0.037) with I and AHI increasing the most (13.6% increase, P=0.010 and 6.7% increase, P=0.040, respectively).

### Tables VII and VIII

Table VII. has the volume fraction of cells and blood vessels for the injected animals at 2 weeks.

TABLE VII
Volume fraction measurements at 2 weeks.

| | | | | Albumin + | Albumin + |
|---|---|---|---|---|---|
| | | | | Rabbit | Human |
| | | | Albumin + | PBMC + | EPC + |
| | Control | Injection | Injection | Injection | Injection |
| BVv - inner | 4.30 ± 0.66 | 4.61 ± 0.87 | 4.30 ± 1.12 | 3.94 ± 1.41 | 5.58 ± 0.92 |
| BVv - outer | 3.70 ± 0.84 | 4.24 ± 1.37 | 4.00 ± 1.04 | 3.27 ± 1.24 | 3.64 ± 2.11 |
| Fv - inner | 23.33 ± 8.86 | 25.58 ± 8.49 | 22.30 ± 6.60 | 24.67 ± 6.86 | 25.82 ± 3.80 |
| Fv - outer | 15.58 ± 4.34 | 20.06 ± 8.42 | 17.76 ± 5.30 | 18.85 ± 5.46 | 17.58 ± 4.93 |
| Mv - inner | 1.29 ± 1.46 | 0.85 ± 0.50 | 1.70 ± 0.55 | 1.33 ± 0.27 | 1.70 ± 0.66 |
| Mv - outer | 1.76 ± 0.54 | 1.52 ± 0.96 | 1.33 ± 0.46 | 1.15 ± 0.33 | 1.21 ± 0.43 |
| Nv - inner | 3.45 ± 1.26 | 3.45 ± 1.57 | 5.27 ± 1.94 | 3.55 ± 1.03 | 5.09 ± 1.27 |
| Nv - outer | 5.76 ± 1.05 | 5.15 ± 3.96 | 4.24 ± 1.60 | 3.82 ± 1.77 | 4.24 ± 0.61 |

Data shown are mean ± standard deviation.

Table VIII. has the volume fraction of cells and blood vessels for the injected animals at 3 weeks.

TABLE VIII
Volume fraction measurements at 3 weeks.

| | | | | Albumin + | Albumin + |
|---|---|---|---|---|---|
| | | | | Rabbit | Human |
| | | | Albumin + | PBMC + | EPC + |
| | Control | Injection | Injection | Injection | Injection |
| BVv - inner | 3.70 ± 1.71 | 3.52 ± 1.17 | 3.39 ± 1.89 | 3.39 ± 1.18 | 3.64 ± 1.36 |
| BVv - outer | 4.12 ± 0.95 | 3.85 ± 1.00 | 3.79 ± 1.54 | 3.64 ± 1.01 | 3.76 ± 1.61 |
| Fv - inner | 52.00 ± 4.60 | 51.61 ± 5.24 | 54.91 ± 2.59 | 49.94 ± 1.54 | 52.24 ± 7.81 |
| Fv - outer | 48.18 ± 6.27 | 49.00 ± 6.39 | 47.58 ± 5.08 | 45.39 ± 5.58 | 47.33 ± 5.31 |
| Mv - inner | 0.67 ± 0.25 | 0.73 ± 0.51 | 0.67 ± 0.40 | 0.91 ± 0.21 | 0.67 ± 0.33 |
| Mv - outer | 0.73 ± 0.35 | 0.58 ± 0.17 | 0.61 ± 0.30 | 0.73 ± 0.41 | 0.73 ± 0.46 |
| Nv - inner | 1.21 ± 0.37 | 1.12 ± 0.40 | 1.15 ± 0.69 | 1.39 ± 0.79 | 1.15 ± 0.45 |
| Nv - outer | 1.39 ± 0.46 | 1.00 ± 0.33 | 1.09 ± 0.46 | 1.27 ± 0.78 | 1.15 ± 0.50 |

Data shown are mean ± standard deviation.

### Blood Vessels (BV)—Tables VII and VIII

At 2 weeks (Table VII), AHI lead to a significantly higher volume fraction of blood vessels (BVᵥ) than C in the inner region (29.8% increase, P=0.036). In the outer region, ARI was significantly lower than 1 (22.9% decrease, P=0.035). There were no differences between treatments in either region at 3 weeks (Table VIII) or between the two regions at either time period. However, there was a decrease in BVᵥ between the 2- and 3-week period in the inner region (21.5% decrease, P=0.015), with the biggest change for AHI (34.8% decrease, P=0.030).

### Fibroblasts (F)—Tables VII and VIII

At 2 weeks (Table VII), there were no significant differences in the volume fraction of fibroblasts (Fᵥ) between treatments in either region. At 3 weeks (Table VIII), ARI produced significantly lower Fᵥ in the inner region that AI (9.1% decrease, P=0.026). Between the two time periods, Fᵥ increased significantly for all treatments in both the inner (115.1% average increase, P<0.001) and outer regions (166.3% average increase, P<0.001). Comparing the inner and outer regions of the wound, there was significantly lower Fᵥ in the outer regions at both 2 weeks (26.1% average decrease, P=0.001) and 3 weeks (8.8% average decrease, P=0.004).

### Macrophages (M)—Tables VII and VIII

At 2 weeks, there was a significant increase in the volume fraction of macrophages (Mᵥ) for AI compared to I in the inner region (100.0% increase, P=0.045). There were no significant differences in the outer region at 2 weeks or in either region at 3 weeks. However, there were significant decreases in the Mᵥ between 2 and 3 weeks for both the inner region (43.0% average decrease, P=0.020) and the outer region (50.1% average decrease, P=0.004). Overall, there the outer region produced significantly higher Mᵥ than the inner region (2.0% average increase, P<0.001).

### Neutrophils (N)

At 2 weeks (Table VII), AHI lead to a significantly lower volume fraction of neutrophils (Nᵥ) than C (26.4% decrease, P=0.023) in the outer region. There were no differences between treatments at 3 weeks (Table VIII). However, there were significant decreases in Nᵥ between the 2- and 3-week periods for both the inner (69.8% average decrease, P=0.001) and outer regions (75.0% average decrease, P=0.001). Overall, the outer region produced higher Nᵥ than the inner region (7.8% average increase, P<0.001). Compared to the non-injected animals, there was a lower Nᵥ in the inner region for the injected animals at 2 weeks (16.1% average decrease, P=0.018).

Any patents or publications mentioned in this specification are incorporated herein by reference to the same extent as if each individual publication is specifically and individually indicated to be incorporated by reference. In addition to other patents and publications cited herein, U.S. Pat. No. 6,656,496 and U.S. Provisional Application Ser. No. 60/673,589 filed Apr. 21, 2005 are hereby incorporated by reference in their entirety.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

What is claimed is:

1. A composition for promoting wound healing at a wound site of a subject, comprising:
a biodegradable scaffold protein that is cross-linkable;
at least one wound healing cell; and
a cross-linking agent that after the composition is administered at the wound site, induces cross-linking of the scaffold protein to form a scaffold having said wound healing cell incorporated within the scaffold.

2. The composition of claim 1 wherein said biodegradable scaffold protein is a plasma protein or an enzymatic modification product thereof.

3. (canceled)

4. The composition of claim 2 wherein said plasma biodegradable scaffold protein is albumin or fibrin.

5-6. (canceled)

7. The composition of claim 2 wherein said biodegradable scaffold protein is isolated from the subject.

8-12. (canceled)

13. The composition of claim 1 wherein said wound healing cell is selected from the group consisting of; an endothelial cell, an inflammatory cell, an epithelial cell, a platelet, a neutrophil, a lymphocyte, a fibroblast, an endothelial cell predecessor, an inflammatory cell predecessor, an epithelial cell predecessor, a platelet predecessor, a neutrophils predecessor, a lymphocyte predecessor and a fibroblast predecessor.

14. (canceled)

15. The composition of claim 13 wherein said wound healing cell type is isolated from the subject.

16-18. (canceled)

19. The composition of claim 13 wherein sad endothelial cell predecessor is isolated from a source selected from the group consisting of: bone marrow, peripheral blood, umbilical cord blood, and fetal liver.

20-22. (canceled)

23. The composition of claim 1 further comprising a bioactive agent selected from the group consisting of: an antibiotic agent, an anti-inflammatory agent, an analgesic agent, an anesthetic agent, a cytokine and a growth factor.

24. (canceled)

25. The composition of claim 1 wherein said wound healing cell comprises an expression construct encoding a bioactive agent selected from the group consisting of: an antibiotic agent, an anti-inflammatory agent, an analgesic agent, an anesthetic agent, a cytokine, and a growth factor.

26-28. (canceled)

29. A process for promoting wound healing at a wound site of a subject, comprising:
treating the wound site with an adjunctive wound healing method selected from the group consisting of electrostimulation, hydrotherapy, hyperbaric oxygen treatment, negative pressure therapy, ultrasound treatment, and a combination thereof; and
administering at the wound site a composition comprising:
a biodegradable scaffold protein that is cross-linkable;
at least one wound healing cell; and
a cross-linking agent that after the composition is administered at the wound site, induces cross-linking of the scaffold protein to form a scaffold having said wound healing cell incorporated within the scaffold.

30-31. (canceled)

32. The process of claim 29 further comprising isolating said wound healing cell from the subject.

33. (canceled)

34. The process of claim 29 further comprising:
isolating said biodegradable scaffold protein from a blood specimen obtained from the subject.

35-37. (canceled)

38. The process of claim 29 further comprising:
disposing a cell comprising an expression construct encoding a bioactive agent on said scaffold.

39-41. (canceled)

42. A process for promoting wound healing, comprising:
providing a wound healing cell that acts prior to said wound healing cell type in wound healing;
providing a cross-linkable proteinaceous component of a biodegradable scaffold;
providing a cross-linker component of a degradable scaffold;
adding the cell to the cross-linkable proteinaceous component to yield a first constituent and/or adding the cell to the cross-linker component to yield a second constituent;
mixing the first constituent and cross-linker component, the second constituent and the cross-linkable component, or the first and second constituents to yield a composition for wound healing; and
applying the composition to a wound of an individual to promote wound healing.

43. The process of claim 42 further comprising administering said wound healing cell independent of said scaffold.

44. The process of claim 43 wherein the administering is by local or systemic injection.

45. The process of claim 42 wherein said wound healing cell is derived from said individual.

46-49. (canceled)

50. The process of claim 42 further comprising an adjunctive wound healing treatment selected from the group consisting of: electrostimulation, hydrotherapy, hyperbaric oxygen treatment, negative pressure therapy, ultrasound treatment and a combination thereof.

51. The composition of claim 1 wherein the biodegradable scaffold protein, the wound healing cell, and the cross-linking agent are mixed and administered in a solution.
